(19) United States
(12) Patent Application Publication
(10) Pub. No.: US 2018/0294417 A1
(43) Pub. Date: Oct. 11, 2018

(54) FLUORENE-CARBAZOLE DERIVATIVES AND PHOSPHORESCENT ORGANIC ELECTROLUMINESCENT DEVICES

(71) Applicant: Wuhan China Star Optoelectronics Technology Co., Ltd., Wuhan, Hubei (CN)

(72) Inventor: Biao PAN, Wuhan (CN)

(21) Appl. No.: 15/567,377

(22) PCT Filed: May 18, 2017
(86) PCT No.: PCT/CN2017/084850

§ 371 Date: Oct. 18, 2017

(30) Foreign Application Priority Data
Jan. 3, 2017 (CN) 201710117746.4

(51) Int. Cl. H01L 51/00; C07F 9/572; C09K 11/06; C07D 401/04; C07D 209/96

(52) U.S. Cl. CPC H01L 51/0072; C07F 9/5728; C09K 11/06; H01L 51/5016; H01L 51/0067; C07D 209/96; C07D 401/04

(57) ABSTRACT

The present disclosure provides fluorene-carbazole derivatives and phosphorescent organic electroluminescent devices using the fluorene-carbazole derivatives. The fluorene-carbazole derivative is represented by General Formula I. In the present disclosure, fluorene-carbazole is the core, spirobifluorene is combined with carbazole to reduce the loss of triplet energy without a change in hole mobility and Tg.

### FIELD OF THE INVENTION

The present disclosure relates to the field of displays, and in particular to fluorene-carbazole derivatives and phosphorescent organic electroluminescent devices.

### BACKGROUND OF THE INVENTION

In 1987, Professors DENG Qingyun and Vanslyke made a bilayer organic electroluminescent device using Ultrathin Film Technology by using a transparent conducting film as an anode, AlQ3 as a luminescent layer, triarylamine as a hole transporting layer, and Mg/Ag alloy as a cathode (Appl. Phys. Lett., 1987, 52, 913). In 1990, Burroughes et al. invented an OLED having a luminescent layer made of conjugated polymers PPV (Nature. 1990, 347, 539). Since then, OLEDs have been a hot research topic all over the world.

Because of the influence of spin-forbidden reactions, most of what we usually see in daily life is a phenomenon of fluorescence. Research on OLED technology was initially focused on fluorescent devices. However, according to the theory of spin quantum statistics, a maximum Internal Quantum Efficiency of a fluorescent electroluminescent device is only 25%, while that of a phosphorescent electroluminescent device can reach 100%. Accordingly, in 1999, Forrest and Thompson et al. (Appl. Phys. Let., 1999, 75, 4.) obtained a green OLED by doping a green phosphorescent material Ir(PPy)3 into a host material of 4,4′-N, N′-dicarbazole-biphenyl (CBP) at a concentration of 6 wt %. The maximum External Quantum Efficiency (EQE) of such a green OLED reaches 8%, which broke the theoretical limit of electroluminescent devices. After that, phosphorescent luminescent materials have generated significant interest. Since then, phosphorescent electroluminescent materials and phosphorescent devices become a hot research topic all over the world.

There are three crucial factors for a good phosphorescent material: the first one is a sufficiently high triplet energy level (ET) to achieve effective energy transfer; the second one is a balanced carrier transporting in devices for enhancing luminous efficiency of the devices; and the last one is a sufficiently high glass transition temperature (Tg) to ensure the stability of the devices at high current density for increasing the life of organic luminescent devices. In order to achieve these three different requirements simultaneously on one molecule, researchers carried out a number of meaningful attempts and developed different types of phosphorescent luminescent host materials.

Carbazole derivatives, such as 1,3-bis(carbazol-9-yl)benzene (mCP), have been widely used in phosphorescent electroluminescent diodes (PHOLEDs) due to their high triplet energy and good hole-transporting capability. However, carbazole derivatives alone do not have a high Tg, so there is a need to combine it with a structure having a high Tg by means of molecular designing. Spirofluorene is one of the few structure units that have a high ET (>2.8 eV) and a high Tg (>150° C.). Therefore, it will be a very effective design for a phosphorescent host material if spirobifluorene is combined with carbazole. However, applications of such molecules are very rare.

As a result, it is necessary to provide a new fluorene-carbazole derivative to solve the problems existing in the conventional technologies.

### SUMMARY OF THE INVENTION

The object of the present disclosure is to provide a new fluorene-carbazole derivative and phosphorescent organic electroluminescent devices using such a new fluorene-carbazole derivative. In the present disclosure, fluorene-carbazole is the core, spirobifluorene is combined with carbazole to reduce the loss of triplet energy without a change in hole mobility or Tg. In addition, various electron-transporting groups are bonded to the fluorene of the spirobifluorene to achieve the balance of carrier transport, so that a kind of bipolar blue-phosphorescent host materials with high triplet, high electron mobility, and high thermal stability is obtained. Applying such host materials to the preparations of high efficient phosphorescent electroluminescent devices solves the problem that the traditional phosphorescent materials cannot simultaneously achieve high triplet energy level, carrier transfer matching, and high glass transition temperature.

To achieve the above objects, the present disclosure first provides a fluorene-carbazole derivative represented by the following General Formula I:

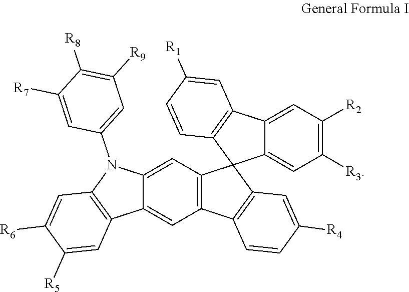

In a preferred embodiment of the present disclosure, R₃ represents diphenylphosphoryl, 3-pyridinyl, or cyano, and each of R₁, R₂, R₄, R₅, R₆, R₇, R₈ and R₉ independently represents hydrogen.

In one embodiment of the present disclosure, R₁, R₂, R₃ and R₄ are electron-transporting groups, and R₅, R₆, R₇, R₈ and R₉ are hole-transporting groups.

In one embodiment of the present disclosure, the electron-transporting group is selected from a group consisting of hydrogen, cyano, diphenylphosphoryl, p-triphenylphosphynyl group, m-triphenylphosphynyl group, o-triphenylphosphynyl group, 2-pyridinyl, 3-pyridinyl, 4-pyridinyl, aza-9-carbazolyl, p-phenyl-benzoimidazolyl group, 4-N-benzimidazle, m-phenyl-benzoimidazolyl group, o-phenyl-benzoimidazolyl group, 3-N-benzimidazle, o-phenyl-1,3,4-oxadiazolyl group, m-phenyl-1,3,4-oxadiazolyl group, p-phenyl-1,3,4-oxadiazolyl group, o-phenyl-1,4,5-triazolyl group, m-phenyl-1,4,5-triazolyl group, p-phenyl-1,4,5-triazolyl group, o-triphenylphosphynyl group, 2-dioxodibenzothiophenyl, 3-dioxodibenzothiophenyl, 4-dioxodibenzothiophenyl, phenanthroimidazolyl, N-phenanthroimidazolyl, and p-phenyl-phenanthroimidazolyl group.

In one embodiment of the present disclosure, the hole-transporting group is selected from a group consisting of hydrogen, phenyl, p-methylphenyl group, 9-carbazolyl, tert-butyl-9-carbazolyl, aza-9-carbazolyl, diaza-9-carbazolyl, triphenylsilyl, p-triphenylamine group, dimethyl-p-triphenylamine group, di-tert-butyl substituted carbazolyl group, 1-naphthyl substituted p-triphenylamine group, 2-naphthyl substituted p-triphenylamine group, 3,6-di-tert-butyl-carbazolylphenyl, bis(3,6-di-tert-butyl-carbazolyl) substituted phenyl group, p-triphenylamine group, dimethyl-p-triphenylamine group, 1-naphthyl substituted p-triphenylamine group, 2-naphthyl substituted p-triphenylamine group, p-carbazolyl-phenyl group, (pyridyl-3-yl)carbazolyl, 2-dibenzothiophene, 3-dibenzothiophene, and 4-dibenzothiophene.

In one embodiment of the present disclosure, R₁, R₂, R₃ and R₄ represent the same or different substituent groups.

In one embodiment of the present disclosure, R₅, R₆, R₇, R8 and R₉ represent the same or different substituent groups.

In a preferred embodiment of the present disclosure, each of R₁, R₂, R₄, R₅, R₆, R₇, R₈ and R₉ independently represents hydrogen.

In a preferred embodiment of the present disclosure, R₃ represents diphenylphosphoryl, 3-pyridinyl, or cyano.

In a preferred embodiment of the present disclosure, a fluorene-carbazole derivative is provided and is represented by the following Formula i, ii, or iii:

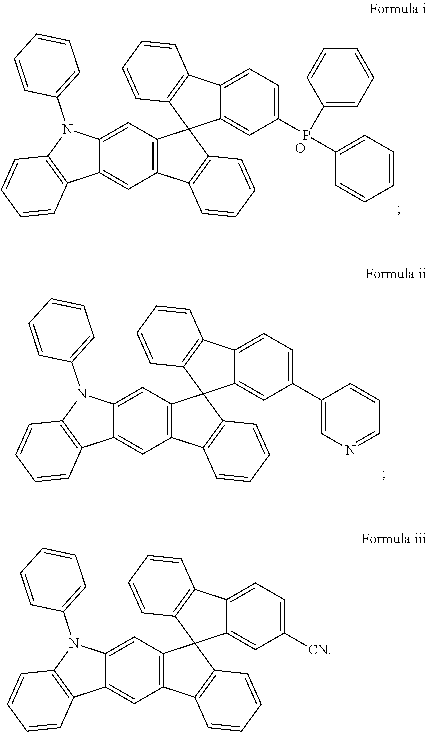

The present disclosure further provides a preparation method of the above fluorene-carbazole derivatives. The preparation method is that an intermediate 1 is obtained by carrying out a Suzuki reaction coupling the starting materials of 3-pinacolatoboryl-9-phenyl-9H-carbazole and m-bromoiodobenzene; then, after a reaction between the intermediate 1 and 3-(2-bromophenyl)-9-phenyl-9H-carbazole under −78° C., a reaction with 2-bromofluorenone is conducted, to obtain 2-bromo-fluorene-carbazole; and finally, the final product is obtained by carrying out a Suzuki reaction, or a cyanation reaction, or a reaction between n-butyllithium and chlorodiphenyl phosphine with hydrogen peroxide.

In a preferred embodiment of the present disclosure, there is provided a preparation method of the above fluorene-carbazole derivative represented by Formula i. The preparation method is that an intermediate 1 is obtained by carrying out a Suzuki reaction coupling the starting materials of 3-pinacolatoboryl-9-phenyl-9H-carbazole and m-bromoiodobenzene; then, after a reaction between the intermediate 1 and 3-(2-bromophenyl)-9-phenyl-9H-carbazole under −78° C., a reaction with 2-bromofluorenone is conducted, to obtain 2-bromo-fluorene-carbazole; and finally, the final product is obtained by reacting 2-bromo-fluorene-carbazole with n-butyllithium, chlorodiphenyl phosphine, and hydrogen peroxide.

In a preferred embodiment of the present disclosure, there is provided a preparation method of the above fluorene-carbazole derivative represented by Formula ii. The preparation method is that an intermediate 1 is obtained by carrying out a Suzuki reaction coupling the starting materials of 3-pinacolatoboryl-9-phenyl-9H-carbazole and m-bromoiodobenzene; then, after a reaction between the intermediate 1 and 3-(2-bromophenyl)-9-phenyl-9H-carbazole under −78° C., a reaction with 2-bromofluorenone is conducted, to obtain 2-bromo-fluorene-carbazole; and finally, the final product is obtained by carrying out a Suzuki reaction using 2-bromo-fluorene-carbazole, 3-pyridylboronic acid, tetrakis(triphenylphosphine)palladium\potassium carbonate, toluene, and absolute ethanol.

In a preferred embodiment of the present disclosure, there is provided a preparation method of the above fluorene-carbazole derivative represented by Formula iii. The preparation method is that an intermediate 1 is obtained by carrying out a Suzuki reaction coupling the starting materials of 3-pinacolatoboryl-9-phenyl-9H-carbazole and m-bromoiodobenzene; then, after a reaction between the intermediate 1 and 3-(2-bromophenyl)-9-phenyl-9H-carbazole under −78° C., a reaction with 2-bromofluorenone is conducted, to obtain 2-bromo-fluorene-carbazole; and finally, the final product is obtained by carrying out a cyanation reaction between 2-bromo-fluorene-carbazole and copper cyanide.

The present disclosure further provides a phosphorescent organic electroluminescent device using the above-mentioned fluorene-carbazole derivatives as a host material. In particular, the phosphorescent organic electroluminescent device provided in the present disclosure comprises at least one organic electroluminescent layer containing the fluorene-carbazole derivative represented by Formula i, ii, or iii.

In one embodiment of the present disclosure, the phosphorescent organic electroluminescent device comprises: a first electrode layer arranged on a substrate; one or more organic electroluminescent layers arranged on the first electrode layer, wherein the organic electroluminescent layer has a thickness of 15 to 25 nm and is made of the spirofluorene derivative doped with FIrpic; and a second electrode layer arranged on the organic electroluminescent layer.

In a preferred embodiment of the present disclosure, the doping ratio of the FIrpic is 5 to 10 wt %, preferably 7 wt %.

In one embodiment of the present disclosure, the phosphorescent organic electroluminescent device further comprises: an electron injecting layer having a thickness of 0.5 to 1.5 nm and sandwiched between the second electrode layer and the organic electroluminescent layer, an electron transporting layer having a thickness of 30 to 50 nm and sandwiched between the electron injecting layer and the organic electroluminescent layer, a hole injecting layer having a thickness of 5 to 15 nm and sandwiched between the first electrode layer and the organic electroluminescent layer, a hole transporting layer having a thickness of 60 to 80 nm and sandwiched between the hole injecting layer and the organic electroluminescent layer, and an exciton blocking layer sandwiched having a thickness of 2 to 10 nm and between the hole transporting layer and the organic electroluminescent layer.

In a preferred embodiment of the present disclosure, the electron injecting layer has a thickness of 0.5 to 1.5 nm, the electron transporting layer has a thickness of 30 to 50 nm, the hole injecting layer has a thickness of 5 to 15 nm, the hole transporting layer has a thickness of 60 to 80 nm, and the exciton blocking layer has a thickness of 2 to 10 nm.

In one embodiment of the present disclosure, the first electrode layer (anode) is formed of ITO, the hole injecting layer is formed of molybdenum trioxide, the hole transporting layer is formed of NPB, the exciton blocking layer is formed of mCP, the electron transporting layer is formed of TmPyPB, the electron injecting layer is formed of LiF, and the second electrode layer (cathode) is formed of Al.

The invention has the following advantages.

(1) The fluorene-carbazole derivatives provided in the present disclosure have a higher triplet energy level to realize the energy transfer of the triplet excitons from the host to the guest.

(2) The fluorene-carbazole derivatives provided in the present disclosure have a balanced carrier mobility to realize an effective recombination of holes and electrons in the light emitting region for increasing the luminous efficiency of the device.

(3) The fluorene-carbazole derivatives provided in the present disclosure have a higher glass transition temperature and a better thermal stability, so that the service life of the light emitting device can be improved.

(4) OLED devices comprising a luminescent layer made of the fluorene-carbazole derivatives according to the present disclosure have an excellent performance, and the current efficiency, the power efficiency, and the external quantum efficiency thereof can achieve the top level in the performance of current blue phosphorescent devices.

(5) OLED devices comprising an electron transporting layer made of the fluorene-carbazole derivatives according to the present disclosure have a good stability within a large voltage range, which may effectively reduce the interfacial energy barrier between the electron transporting layer and the luminescent layer, avoid the interfacial charge accumulation and exciton quenching, and help to increase the lifetime of devices, so that the OLED devices have a wide application prospect in the full color display field.

### BRIEF DESCRIPTION OF THE DRAWINGS

The above and other objects, features, and advantages of the present disclosure will be more apparent from the following detailed description taken in conjunction with the accompanying drawings, in which:

FIG. 1 is a UV absorption spectrum, a fluorescence emission spectrum, and a low-temperature phosphorescence spectrum of the fluorene-carbazole derivative SPDPO according to one embodiment of the present disclosure. FIG. 1 illustrates that the maximum UV absorption peak of SPDPO is near 290 nm.

FIG. 2 is a structural schematic view of a phosphorescent organic electroluminescent device according to one embodiment of the present disclosure.

FIG. 3 is a diagram illustrating the energy level of a phosphorescent organic electroluminescent device according to one embodiment of the present disclosure.

FIG. 4 is a brightness-current density-voltage characteristic curve graph of the phosphorescent organic electroluminescent devices according to one embodiment of the present disclosure.

FIG. 5 is a current efficiency-brightness characteristic curve graph of the phosphorescent organic electroluminescent devices according to one embodiment of the present disclosure.

FIG. 6 is a power efficiency-brightness characteristic curve graph of the phosphorescent organic electroluminescent devices according to one embodiment of the present disclosure.

FIG. 7 is an electroluminescent spectrum of the phosphorescent organic electroluminescent devices according to one embodiment of the present disclosure.

### DESCRIPTION OF THE INVENTION

Hereinafter, some embodiments will be described in detail with reference to illustrative drawings. In the description of the elements of the present disclosure, terms such as “first”, “second”, “A”, “B”, “(a)”, “(b)”, and the like may be used. These terms are merely used to distinguish one component from other components, and the property, order, sequence, and the like of the corresponding component are not limited by the corresponding term. It should be noted that when it is described in the specification that one component is “connected”, “coupled”, or “joined” to another component, a third component may be “connected”, “coupled”, or “joined” between the first and second components, although the first component may be directly connected, coupled, or joined to the second component.

### Example 1. Spirofluorene Derivatives

A fluorene-carbazole derivative is provided in the present embodiment, which is represented by the following General Formula I:

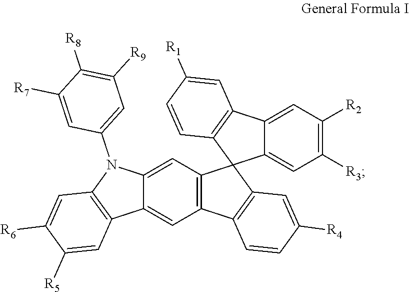

wherein R₁, R₂, R₃ and R₄ are electron-transporting groups, and R₅, R₆, R₇, R₈ and R₉ are hole-transporting groups.

The electron-transporting group includes, but is not limited to, hydrogen, cyano, diphenylphosphoryl, p-triphenylphosphynyl group, m-triphenylphosphynyl group, o-triphenylphosphynyl group, 2-pyridinyl, 3-pyridinyl, 4-pyridinyl, aza-9-carbazolyl, p-phenyl-benzoimidazolyl group, 4-N-benzimidazle, m-phenyl-benzoimidazolyl group, o-phenyl-benzoimidazolyl group, 3-N-benzimidazle, o-phenyl-1,3,4-oxadiazolyl group, m-phenyl-1,3,4-oxadiazolyl group, p-phenyl-1,3,4-oxadiazolyl group, o-phenyl-1,4,5-triazolyl group, m-phenyl-1,4,5-triazolyl group, p-phenyl-1,4,5-triazolyl group, o-triphenylphosphynyl group, 2-dioxodibenzothiophenyl, 3-dioxodibenzothiophenyl, 4-dioxodibenzothiophenyl, phenanthroimidazolyl, N-phenanthroimidazolyl, and p-phenyl-phenanthroimidazolyl group.

The specific structures and names of parts of the above-mentioned electron transport groups are listed below.

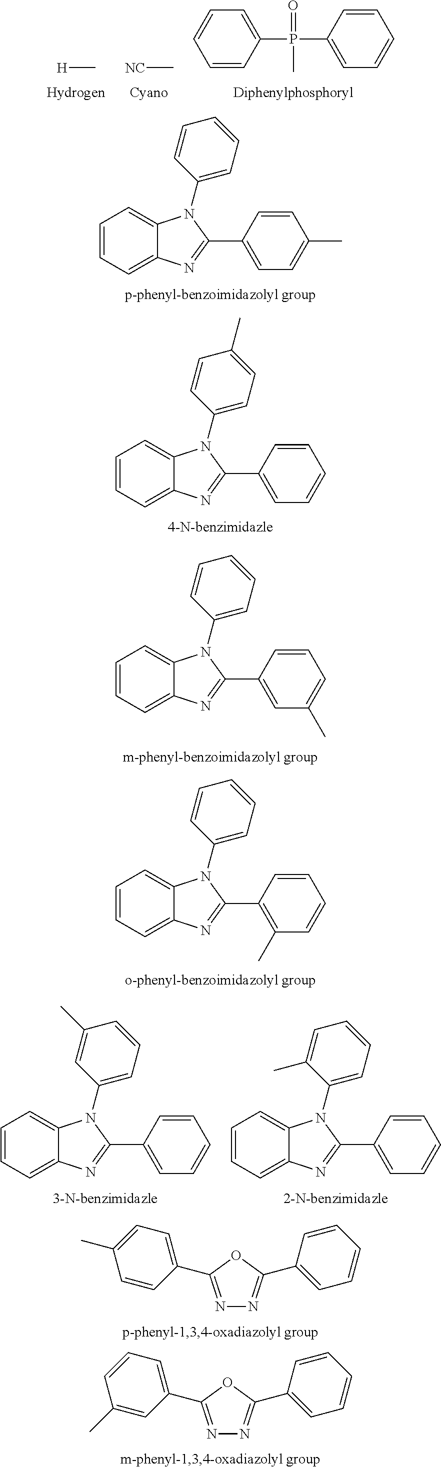

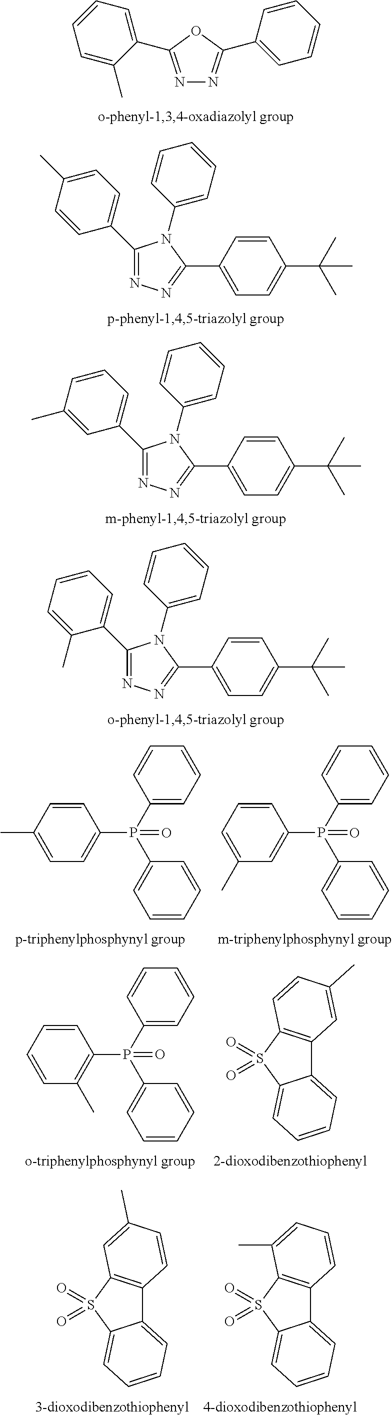

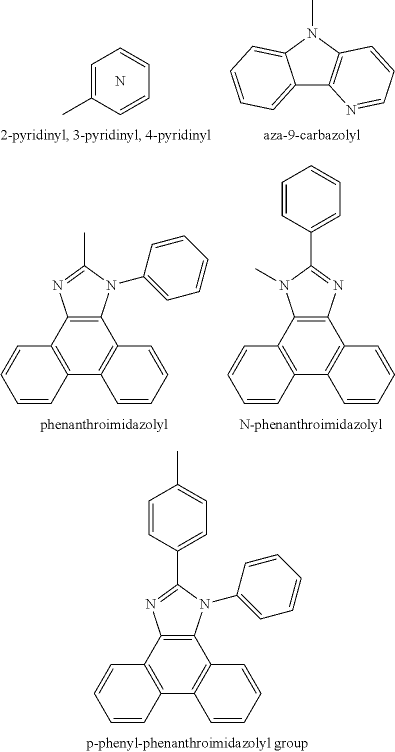

The hole-transporting group includes, but is not limited to, hydrogen, phenyl, p-methylphenyl group, 9-carbazolyl, tert-butyl-9-carbazolyl, aza-9-carbazolyl, diaza-9-carbazolyl, triphenylsilyl, p-triphenylamine group, dimethyl-p-triphenylamine group, di-tert-butyl substituted carbazolyl group, 1-naphthyl substituted p-triphenylamine group, 2-naphthyl substituted p-triphenylamine group, 3,6-di-tert-butyl-carbazolylphenyl, bis(3,6-di-tert-butyl-carbazolyl) substituted phenyl group, p-triphenylamine group, dimethyl-p-triphenylamine group, 1-naphthyl substituted p-triphenylamine group, 2-naphthyl substituted p-triphenylamine group, p-carbazolyl-phenyl group, (pyridyl-3-yl)carbazolyl, 2-dibenzothiophene, 3-dibenzothiophene, and 4-dibenzothiophene.

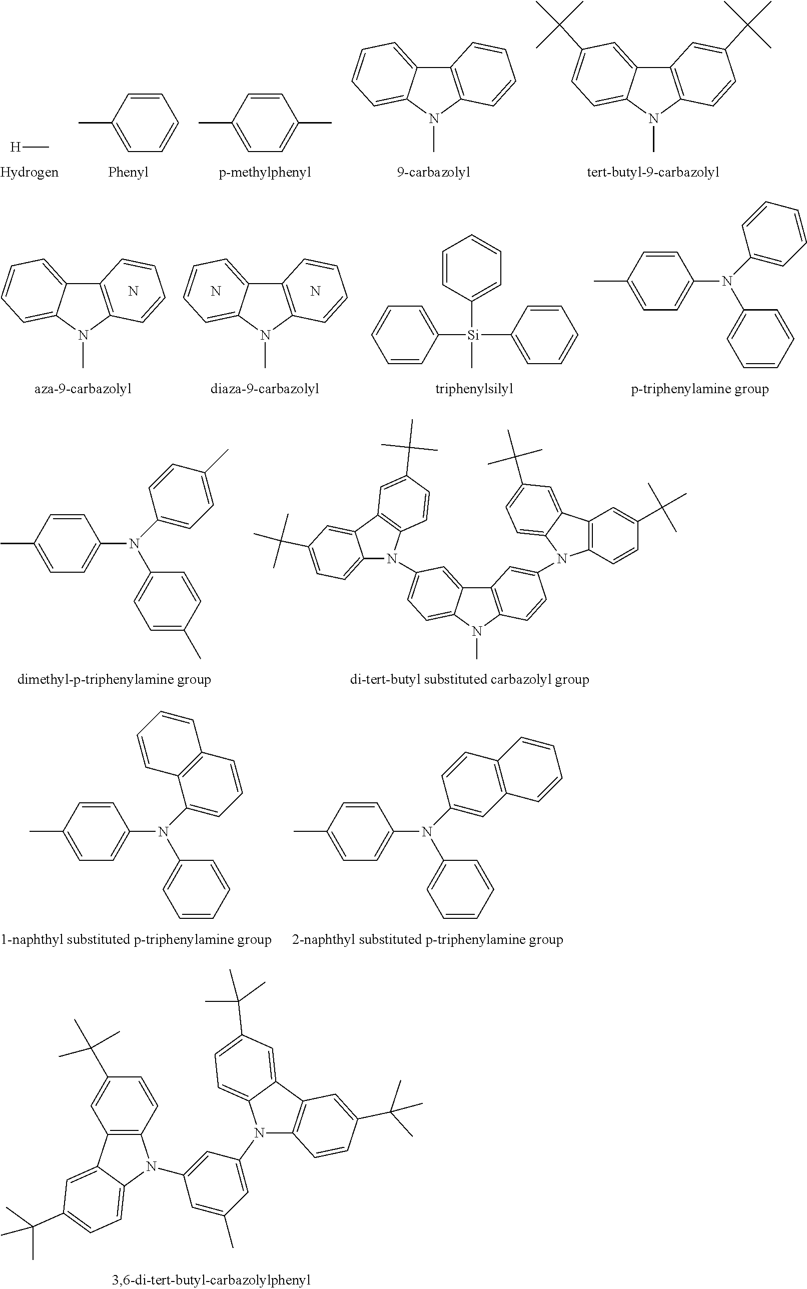

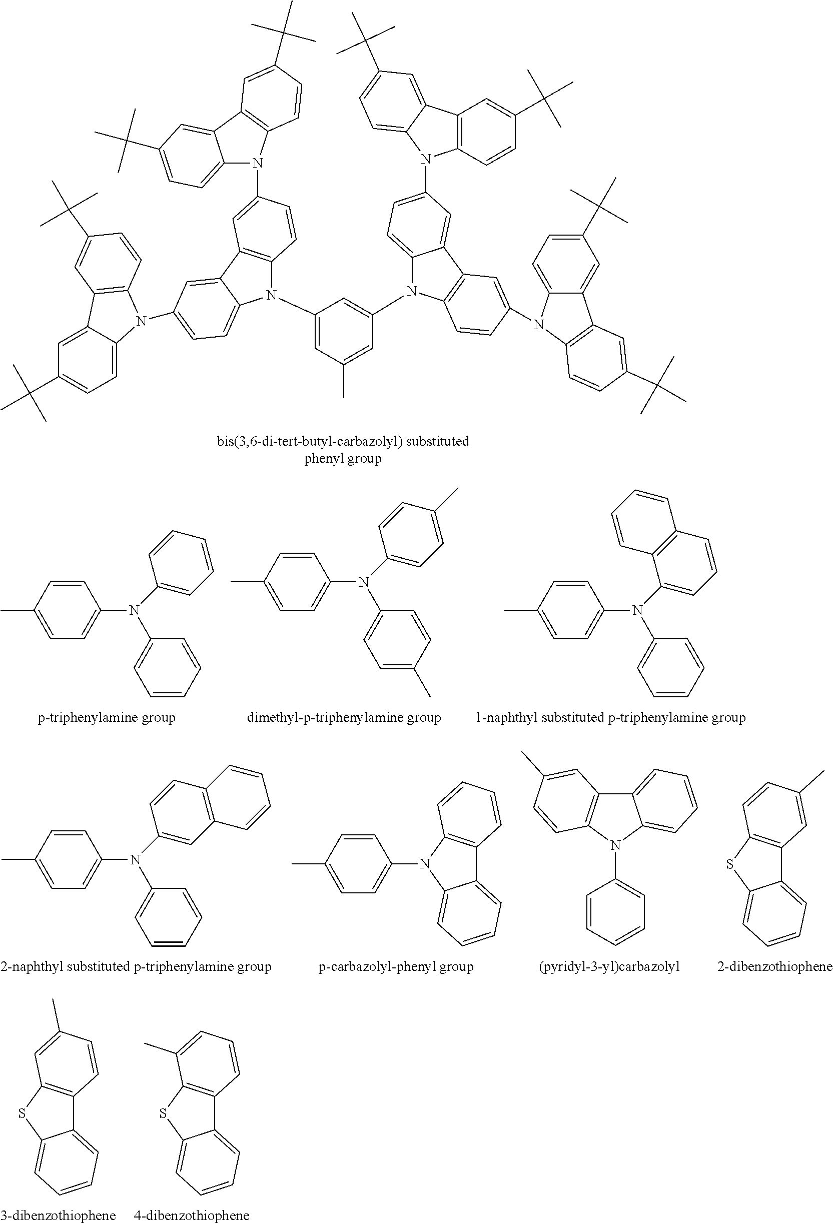

Optionally, each of R₁, R₂, R₄, R₅, R₆, R₇, R₈ and R₉ independently represents hydrogen, while R₃ represents diphenylphosphoryl, 3-pyridinyl, or cyano.

### Example 2. Fluorene-Carbazole Derivative SPDPO

A fluorene-carbazole derivative is provided in the present embodiment, which is represented by Formula i and is denoted as SPDPO:

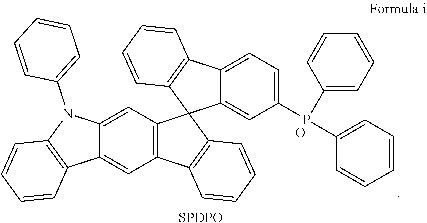

The preparation method is as follows.

### Step 1. Preparation of Intermediate 3-(2-bromophenyl)-9-phenyl-9H-carbazole

1.0 g (2.5 mmol) of 3-pinacolatoboryl-9-phenyl-9H-carbazole, 1.1 g (3.0 mmol) of m-bromoiodobenzene, 0.3 g (0.3 mmol) Pd(PPh3), 5.0 ml of potassium carbonate with a concentration of 2.0 mol/L, 50 ml of toluene and 25 ml of ethanol were successively added into a 150 ml flask to react under nitrogen at 100° C. for 12 hours. The reaction solution was cooled to room temperature followed by being extracted three times with dichloromethane to obtain an organic phase, which was washed three times with water and dried over anhydrous sodium sulfate followed by being filtered, to thereby obtain a crude product after the organic solvent was distilled out. 0.83 g of white solid powder were obtained after purifying the crude product by flash chromatographic column chromatography, that is 3-(2-bromophenyl)-9-phenyl-9H-carbazole.

### Step 2. Preparation of Intermediate 2-bromo-fluorene-carbazole

4.00 g of 3-(2-bromophenyl)-9-phenyl-9H-carbazole obtained in Step 1 was added into 100 ml of dry Tetrahydrofuran (THF), 6 ml of n-butyllithium with a concentration of 2.0 mol/L was slowly added when the reaction solution was cooled to −78° C. to react for 1 hour. Subsequently, 1.8 g of 2-bromofluorenone was added into the reaction solution to react for 2 hours followed by reacting for one night at room temperature. The reaction solution was extracted with dichloromethane, and a crude product was obtained after the solvent was distilled out. The crude product was dissolved in 4 ml of HCl with a concentration of 2.0 mol/L followed by refluxing over night to obtain 2.96 g of product, that is 2-bromo-fluorene-carbazole.

### Step 3. Preparation of the Final Product SPDPO

THF and 2-bromo-fluorene-carbazole obtained in Step 2 were added into a reaction flask under −78° C. followed by adding n-butyllithium dropwise to react at −78° C. for 1 hour. Then, chlorodiphenyl phosphine was added into the reaction solution followed by being heated to room temperature to react for 4 hours. The final product SPDPO was obtained by column chromatography. 1H NMR (DMSO-d6, 400 MHz): δ (ppm) 8.24-8.22 (t, J=8.0 Hz, 2H), 8.19-8.15 (t, J=8.0 Hz, 3H), 7.86-7.82 (t, J=8.0 Hz, 1H), 7.77-7.69 (m, J=8.0 Hz, 3H), 7.49-7.45 (t, J=8.0 Hz, 1H), 7.35-7.21 (m, J=8.0 Hz, 9H), 7.10-7.08 (d, J=8.0 Hz, 1H), 6.92-6.90 (d, J=8.0 Hz, 2H), 6.64-6.62 (t, J=8.0 Hz, 1H); 13C NMR (100 MHz, DMSO-d6): δ (ppm) 180.97, 168.69, 142.17, 140.70, 136.08, 135.99, 132.05, 132.14, 131.70, 131.17, 130.47, 129.51, 129.41, 129.18, 128.72, 126.35, 125.78, 124.50, 123.35, 120.78, 120.42, 119.89, 110.00, 109.72; MS (APCI) (m/z): [M+H+]. The calculation result is C38H24N4S, 568.1, while the measured result is 569.5; and, the calculation result is C38H24N4S: C, 80.26, H, 4.25, N, 9.85, S 5.64, while the measured result is C, 80.26, H, 4.25, N, 9.85, S 5.64.

### Example 3. Fluorene-Carbazole Derivative SPPy

A fluorene-carbazole derivative is provided in the present embodiment, which is represented by Formula ii and is denoted as SPPy:

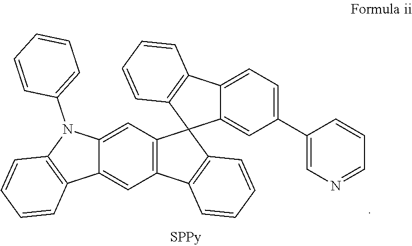 [SMILES: c1ccc(-n2c3ccccc3c3cc4c(cc32)C2(c3ccccc3-c3ccc(-c5cccnc5)cc32)c2ccccc2-4)cc1]

The preparation method is as follows.

### Step 1. Preparation of Intermediate 3-(2-bromophenyl)-9-phenyl-9H-carbazole

1.0 g (2.5 mmol) of 3-pinacolatoboryl-9-phenyl-9H-carbazole, 1.1 g (3.0 mmol) of m-bromoiodobenzene, 0.3 g (0.3 mmol) Pd(PPh3), 5.0 ml of potassium carbonate with a concentration of 2.0 mol/L, 50 ml of toluene and 25 ml of ethanol were successively added into a 150 ml flask to react under nitrogen at 100° C. for 12 hours. The reaction solution was cooled to room temperature followed by being extracted three times with dichloromethane to obtain an organic phase, which was washed three times with water and dried over anhydrous sodium sulfate followed by being filtered, to thereby obtain a crude product after the organic solvent was distilled out. 0.83 g of white solid powder were obtained after purifying the crude product by flash chromatographic column chromatography, that is 3-(2-bromophenyl)-9-phenyl-9H-carbazole.

### Step 2. Preparation of Intermediate 2-bromo-fluorene-carbazole

4.00 g of 3-(2-bromophenyl)-9-phenyl-9H-carbazole obtained in Step 1 was added into 100 ml of dry Tetrahydrofuran (THF), 6 ml of n-butyllithium with a concentration of 2.0 mol/L was slowly added when the reaction solution was cooled to −78° C. to react for 1 hour. Subsequently, 1.8 g of 2-bromofluorenone was added into the reaction solution to react for 2 hours followed by reacting for one night at room temperature. The reaction solution was extracted with dichloromethane, and a crude product was obtained after the solvent was distilled out. The crude product was dissolved in 4 ml of HCl with a concentration of 2.0 mol/L followed by refluxing over night to obtain 2.96 g of product, that is 2-bromo-fluorene-carbazole. Yield is 83%.

### Step 3. Preparation of the Final Product SPPy

2-bromo-fluorene-carbazole obtained in Step 2, 3-pyridylboronic acid, tetrakis(triphenylphosphine)palladium\potassium carbonate, toluene and absolute ethanol were added in a dry flask in turn. The reaction was carried out at a temperature of 100° C. for 12 hours after bubbling with nitrogen for 20 minutes. The reaction solution was cooled to room temperature followed by being extracted three times with dichloromethane to obtain an organic phase, which was washed three times with water and dried over anhydrous sodium sulfate followed by being filtered, to thereby obtain a crude product after the organic solvent was distilled out. White solid powders were obtained after purifying the crude product by flash chromatographic column chromatography, that is the final product SPPy.

### Example 4. Fluorene-Carbazole Derivative SPCN

A fluorene-carbazole derivative is provided in the present embodiment, which is represented by Formula iii and is denoted as SPCN:

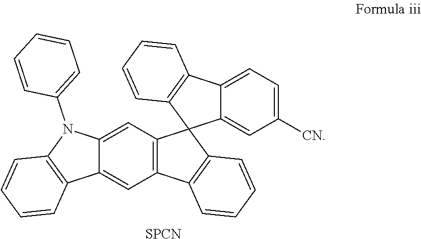

The preparation method is as follows.

### Step 1. Preparation of Intermediate 3-(2-bromophenyl)-9-phenyl-9H-carbazole

1.0 g (2.5 mmol) of 3-pinacolatoboryl-9-phenyl-9H-carbazole, 1.1 g (3.0 mmol) of m-bromoiodobenzene, 0.3 g (0.3 mmol) Pd(PPh3), 5.0 ml of potassium carbonate with a concentration of 2.0 mol/L, 50 ml of toluene and 25 ml of ethanol were successively added into a 150 ml flask to react under nitrogen at 100° C. for 12 hours. The reaction solution was cooled to room temperature followed by being extracted three times with dichloromethane to obtain an organic phase, which was washed three times with water and dried over anhydrous sodium sulfate followed by being filtered, to thereby obtain a crude product after the organic solvent was distilled out. 0.83 g of white solid powder were obtained after purifying the crude product by flash chromatographic column chromatography, that is 3-(2-bromophenyl)-9-phenyl-9H-carbazole.

### Step 2. Preparation of Intermediate 2-bromo-fluorene-carbazole

4.00 g of 3-(2-bromophenyl)-9-phenyl-9H-carbazole obtained in Step 1 was added into 100 ml of dry Tetrahydrofuran (THF), 6 ml of n-butyllithium with a concentration of 2.0 mol/L was slowly added when the reaction solution was cooled to −78° C. to react for 1 hour. Subsequently, 1.8 g of 2-bromofluorenone was added into the reaction solution to react for 2 hours followed by reacting for one night at room temperature. The reaction solution was extracted with dichloromethane, and a crude product was obtained after the solvent was distilled out. The crude product was dissolved in 4 ml of HCl with a concentration of 2.0 mol/L followed by refluxing over night to obtain 2.96 g of product, that is 2-bromo-fluorene-carbazole. Yield is 83%.

### Step 3. Preparation of the Final Product SPCN

Copper cyanide and 2-bromo-fluorene-carbazole obtained in Step 2 were dissolved in DMF to react at 150° C. for 24 hours. The reaction solution was cooled to room temperature and filtered directly. Subsequently, the filter cake was washed with dichloromethane to obtain an organic phase, which was washed three times with water and dried over anhydrous sodium sulfate followed by being filtered, to thereby obtain a crude product after the organic solvent was distilled out. White solid powders were obtained after purifying the crude product by flash chromatographic column chromatography, that is the final product SPCN.

### Example 5. Properties of the Fluorene-Carbazole Derivative SPDPO

The Applicant studied the properties of the fluorene-carbazole derivative SPDPO of the Example 2, and obtained a UV absorption spectrum, a fluorescence emission spectrum, and a low-temperature phosphorescence spectrum as shown in FIG. 1.

FIG. 1 illustrates that the maximum UV absorption peak of SPDPO is near 290 nm, the maximum fluorescence emission peak is at 380 nm, and the triplet energy level is 2.78 eV.

### Example 6. Phosphorescent Organic Electroluminescent Device A

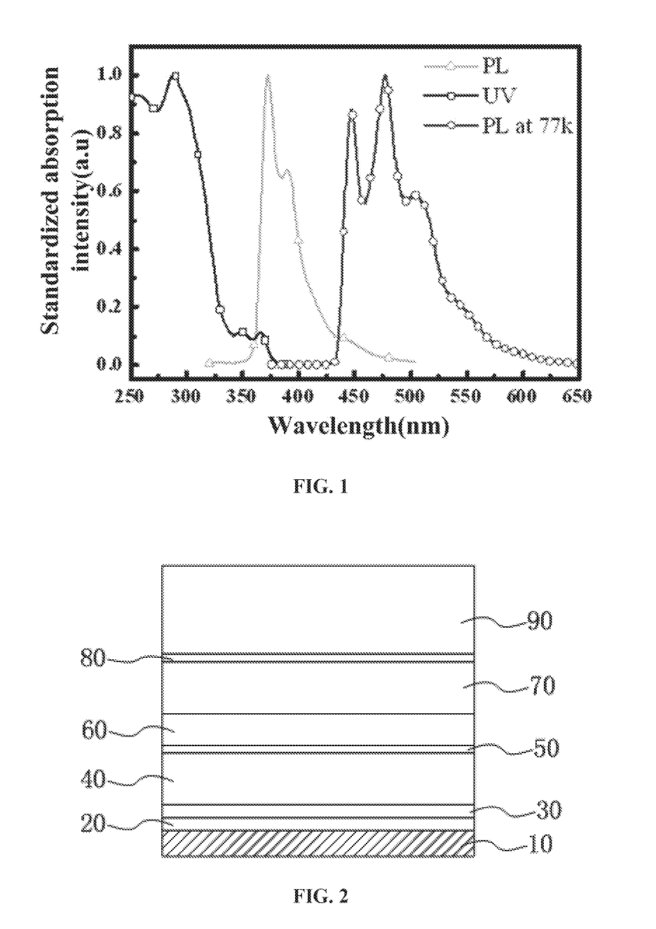

Referring now to FIG. 2, a phosphorescent organic electroluminescent device A is provided in the present embodiment, which comprises: a first electrode layer 20 arranged on a substrate 10, a hole injecting layer 30 arranged on the first electrode layer 20, a hole transporting layer 40 arranged on the hole injecting layer 30, an exciton blocking layer 50 arranged on the hole transporting layer 40, a phosphorescent organic electroluminescent layer 60 arranged on the exciton blocking layer 50 and made of the fluorene-carbazole derivative SPDPO doped with FIrpic, an electron transporting layer 70 arranged on the organic electroluminescent layer 60, an electron injecting layer 80 arranged on the electron transporting layer 70, and a second electrode layer 90 arranged on the electron injecting layer 80.

The doping ratio of the FIrpic is 7 wt % in the present embodiment.

In the present embodiment, the first electrode layer 20(anode) is formed of ITO, the hole injecting layer 30 is formed of molybdenum trioxide(MoO3), the hole transporting layer 40 is formed of NPB, the exciton blocking layer 50 is formed of mCP, the electron transporting layer 70 is formed of TmPyPB, the electron injecting layer 80 is formed of LiF, and the second electrode layer 90 (cathode) is formed of Al.

In the present embodiment, the hole injecting layer 30 has a thickness of 10 nm, the hole transporting layer 40 has a thickness of 70 nm, the exciton blocking layer 50 has a thickness of 5 nm, the organic electroluminescent layer 60 has a thickness of 20 nm, the electron transporting layer 70 has a thickness of 40 nm, the electron injecting layer 80 has a thickness of 1 nm, and the second electrode layer 90 has a thickness of 100 nm.

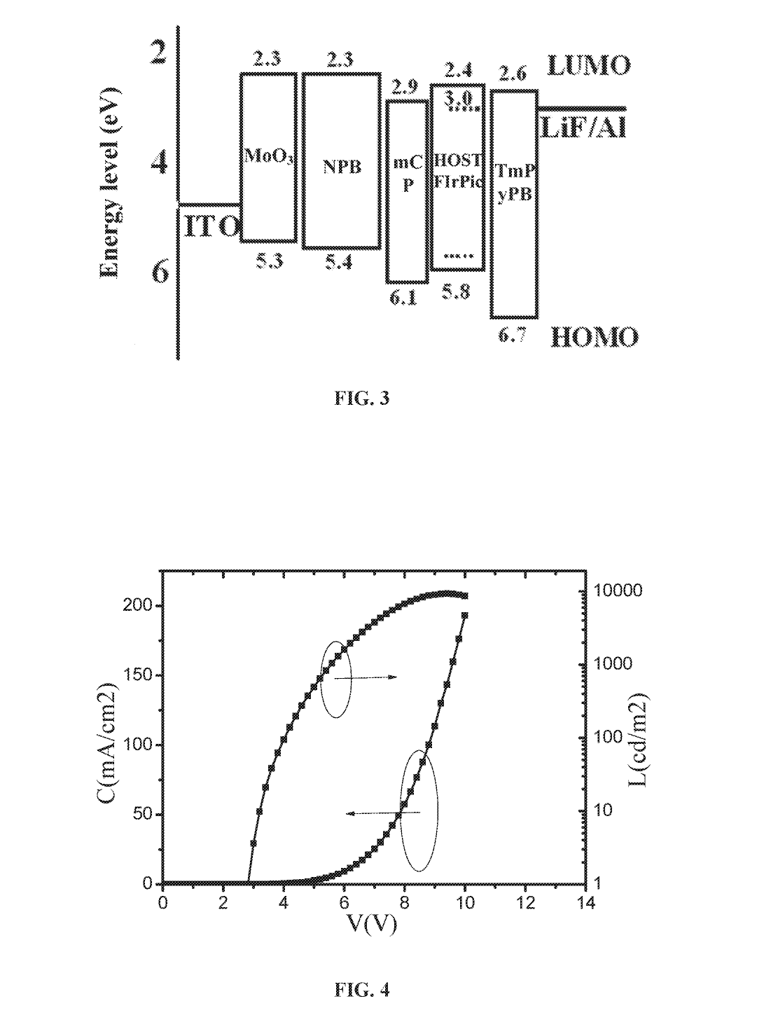

Accordingly, the device structure of the phosphorescent organic electroluminescent device A in the present embodiment is as follows: ITO/MoO3 (10 nm)/NPB (70 nm)/mCP (5 nm)/SPDPO-FIrpic (20 nm)/TmPyPB (40 nm)/LiF (1 nm)/Al (100 nm). A diagram illustrating the energy level is shown in FIG. 3.

The phosphorescent organic electroluminescent device A is prepared by a known method. Such as, but not limited to, a method of cleaning an ITO glass for 30 minutes in both a cleaning agent and deionized water followed by drying in vacuum for 2 hours (105° C.), and then putting the ITO glass into a plasma reactor for CFx plasma treatment for 1 minute followed by transferring it to a vacuum chamber to prepare an organic film and a metal electrode. The SPDPO is prepared as a host material by vacuum deposition for preparing the device.

### Example 7. Phosphorescent Organic Electroluminescent Device B

A phosphorescent organic electroluminescent device B is provided in the present embodiment, which has a similar structure to the phosphorescent organic electroluminescent device A described in Example 6 and the difference is that the organic electroluminescent layer of the phosphorescent organic electroluminescent device B is made of the fluorene-carbazole derivative SPPy doped with FIrpic.

Accordingly, the device structure of the organic electroluminescent device B in the present embodiment is as follows: no/MoO3 (10 nm)/NPB (70 nm)/mCP (5 nm)/SPPy-FIrpic (20 nm)/TmPyPB (40 nm)/LiF (1 nm)/Al.

The phosphorescent organic electroluminescent device B is prepared by a known method. Such as, but not limited to, a method of cleaning a ITO glass for 30 minutes in both a cleaning agent and deionized water followed by drying in vacuum for 2 hours (105 V), and then putting the ITO glass into a plasma reactor for CFx plasma treatment for 1 minute followed by transferring it to a vacuum chamber to prepare an organic film and a metal electrode. The SPPy is prepared as a host material by vacuum deposition for preparing the device.

### Example 8. Phosphorescent Organic Electroluminescent Device C

A phosphorescent organic electroluminescent device C is provided in the present embodiment, which has a similar structure to the phosphorescent organic electroluminescent device A described in Example 6 and the difference is that the organic electroluminescent layer of the phosphorescent organic electroluminescent device C is made of the fluorene-carbazole derivative SPCN doped with FIrpic.

Accordingly, the device structure of the phosphorescent organic electroluminescent device C in the present embodiment is as follows: ITO/MoO3 (10 nm)/NPB (40 nm)/mCP (5 nm)/SPCN-FIrpic (20 nm)/TmPyPB (40 nm)/LiF (1 nm)/Al.

The phosphorescent organic electroluminescent device C is prepared by a known method. Such as, but not limited to, a method of cleaning a ITO glass for 30 minutes in both a cleaning agent and deionized water followed by drying in vacuum for 2 hours (105 V), and then putting the ITO glass into a plasma reactor for CFx plasma treatment for 1 minute followed by transferring it to a vacuum chamber to prepare an organic film and a metal electrode. The SPCN is prepared as a host material by vacuum deposition for preparing the device.

### Example 9. Performance Verification of the Phosphorescent Organic Electroluminescent Devices

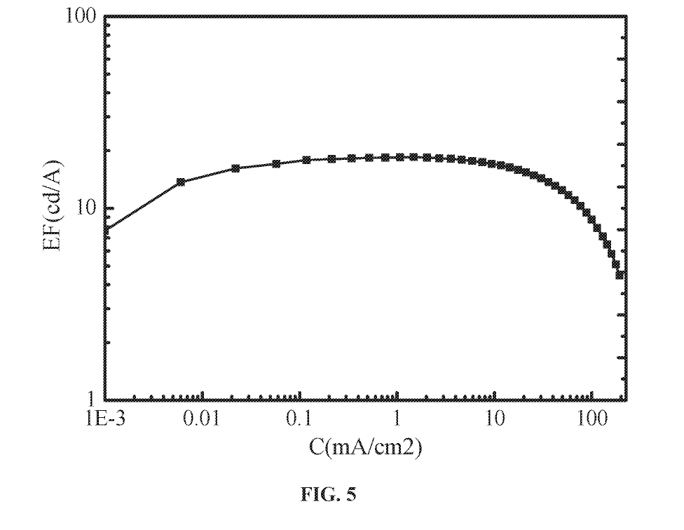

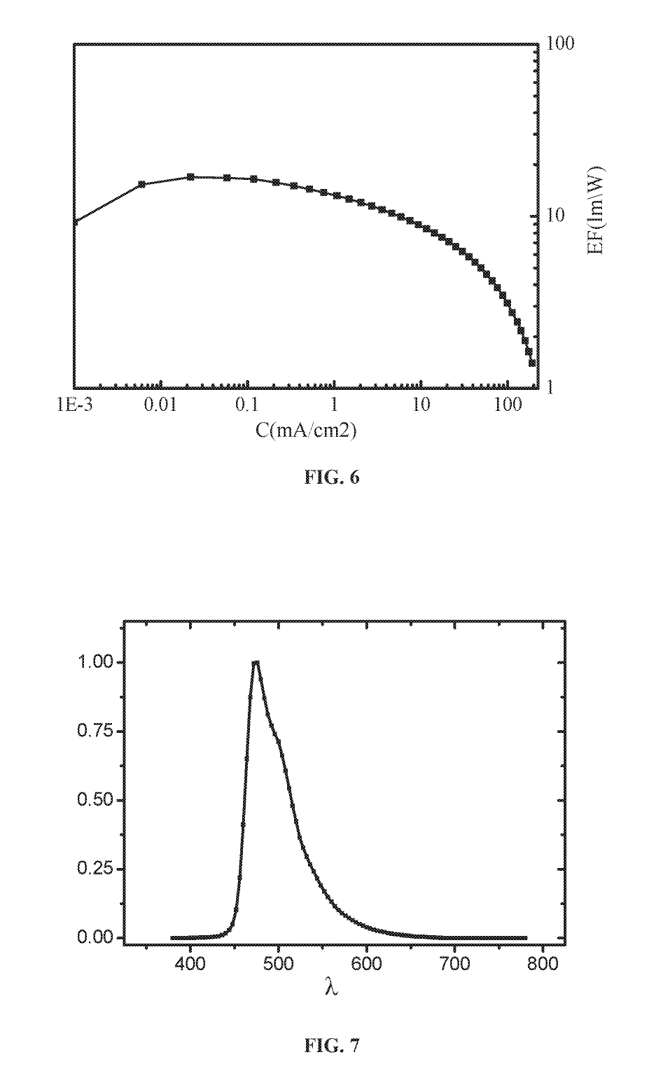

The Applicant further performed performance verification of the phosphorescent organic electroluminescent device A obtained in Example 6, and obtained a brightness-current density-voltage characteristic curve graph as shown in FIG. 4, a current efficiency-brightness characteristic curve graph as shown in FIG. 5, a power efficiency-brightness characteristic curve graph as shown in FIG. 6, and an electroluminescent spectrum as shown in FIG. 7.

FIG. 4 illustrates that the threshold voltage of the phosphorescent organic electroluminescent device A is 2.7 V, which is close to the theoretical minimum threshold voltage.

FIG. 5 illustrates that the maximum current efficiency of the phosphorescent organic electroluminescent device A reaches more than 20 cd/A.

FIG. 6 illustrates that the maximum current efficiency of the phosphorescent organic electroluminescent device A reaches more than 20 lm/W.

FIG. 6 illustrates that in the electroluminescence spectra of the phosphorescent organic electroluminescent device A, only two emission peaks were found at 476 nm and 500 nm, which were characteristic emission peaks of the guest material FIrpic. This shows that triplet excitons are completely transferred.

Thus, the present disclosure has the following advantages.

(1) The fluorene-carbazole derivatives provided in the present disclosure have a higher triplet energy level to realize the energy transfer of the triplet excitons from the host to the guest.

(2) The fluorene-carbazole derivatives provided in the present disclosure have a balanced carrier mobility to realize an effective recombination of holes and electrons in the light emitting region for increasing the luminous efficiency of the device.

(3) The fluorene-carbazole derivatives provided in the present disclosure have a higher glass transition temperature and a better thermal stability, so that the service life of the light emitting device can be improved.

(4) OLED devices comprising a luminescent layer made of the fluorene-carbazole derivatives according to the present disclosure have an excellent performance, and the current efficiency, the power efficiency and the external quantum efficiency thereof can achieve the top level in the performance of current blue phosphorescent devices.

(5) OLED devices comprising an electron transporting layer made of the fluorene-carbazole derivatives according to the present disclosure have a good stability within a large voltage range, which may effectively reduce the interfacial energy barrier between the electron transporting layer and the luminescent layer, avoid the interfacial charge accumulation and exciton quenching and help to increase the lifetime of devices, so that the OLED devices have a wide application prospect in the full color display field.

The present disclosure has been described with relative embodiments which are examples of the present disclosure only. It should be noted that the embodiments disclosed are not the limit of the scope of the present disclosure. Conversely, modifications to the scope and the spirit of the claims, as well as the equal of the claims, are within the scope of the present disclosure.

What is claimed is:

1. A fluorene-carbazole derivative represented by the following General Formula I: 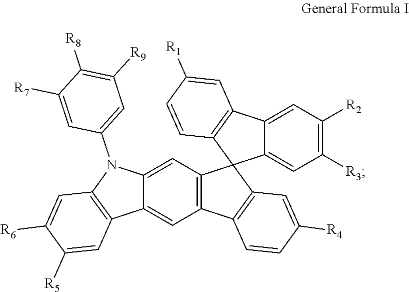
wherein R₃ represents diphenylphosphoryl, 3-pyridinyl, or cyano, and each of R₁, R₂, R₄, R₅, R₆, R₇, R₈ and R₉ independently represents hydrogen.

2. A fluorene-carbazole derivative represented by the following General Formula I: 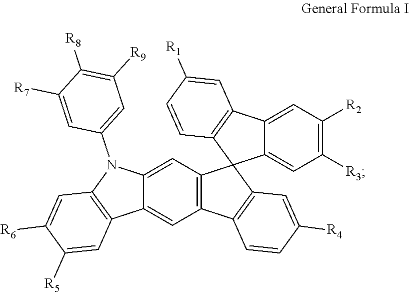
wherein R₁, R₂, R₃ and R₄ are electron-transporting groups, and R₅, R₆, R₇, R₈ and R₉ are hole-transporting groups.

3. The fluorene-carbazole derivative according to claim 1, wherein the electron-transporting group is selected from a group consisting of hydrogen, cyano, diphenylphosphoryl, p-triphenylphosphynyl group, m-triphenylphosphynyl group, o-triphenylphosphynyl group, 2-pyridinyl, 3-pyridinyl, 4-pyridinyl, aza-9-carbazolyl, p-phenyl-benzoimidazolyl group, 4-N-benzimidazle, m-phenyl-benzoimidazolyl group, o-phenyl-benzoimidazolyl group, 3-N-benzimidazle, o-phenyl-1,3,4-oxadiazolyl group, m-phenyl-1,3,4-oxadiazolyl group, p-phenyl-1,3,4-oxadiazolyl group, o-phenyl-1,4,5-triazolyl group, m-phenyl-1,4,5-triazolyl group, p-phenyl-1,4,5-triazolyl group, o-triphenylphosphynyl group, 2-dioxodibenzothiophenyl, 3-dioxodibenzothiophenyl, 4-dioxodibenzothiophenyl, phenanthroimidazolyl, N-phenanthroimidazolyl, and p-phenyl-phenanthroimidazolyl group.

4. The fluorene-carbazole derivative according to claim 3, wherein the hole-transporting group is selected from a group consisting of hydrogen, phenyl, p-methylphenyl group, 9-carbazolyl, tert-butyl-9-carbazolyl, aza-9-carbazolyl, diaza-9-carbazolyl, triphenylsilyl, p-triphenylamine group, dimethyl-p-triphenylamine group, di-tert-butyl substituted carbazolyl group, 1-naphthyl substituted p-triphenylamine group, 2-naphthyl substituted p-triphenylamine group, 3,6-di-tert-butyl-carbazolylphenyl, bis(3,6-di-tert-butyl-carbazolyl) substituted phenyl group, p-triphenylamine group, dimethyl-p-triphenylamine group, 1-naphthyl substituted p-triphenylamine group, 2-naphthyl substituted p-triphenylamine group, p-carbazolyl-phenyl group, (pyridyl-3-yl)carbazolyl, 2-dibenzothiophene, 3-dibenzothiophene, and 4-dibenzothiophene.

5. The fluorene-carbazole derivative according to claim 4, wherein R₁, R₂, R₃ and R₄ are the same or different substituent groups.

6. The fluorene-carbazole derivative according to claim 4, wherein R₅, R₆, R₇, R₈ and R₉ are the same or different substituent groups.

7. The fluorene-carbazole derivative according to claim 4, wherein each of R₁, R₂, R₄, R₅, R₆, R₇, R₈ and R₉ independently represents hydrogen.

8. The fluorene-carbazole derivative according to claim 7, wherein R₃ represents diphenylphosphoryl, 3-pyridinyl, or cyano.

9. A phosphorescent organic electroluminescent device comprising the fluorene-carbazole derivative of claim 1 as a host material.

10. The phosphorescent organic electroluminescent device according to claim 9, wherein the phosphorescent organic electroluminescent device comprises:
a first electrode layer arranged on a substrate,
one or more organic electroluminescent layers arranged on the first electrode layer, wherein the organic electroluminescent layer has a thickness of 15 to 25 nm and is made of the fluorene-carbazole derivative doped with FIrpic, and
a second electrode layer arranged on the organic electroluminescent layer.

11. The phosphorescent organic electroluminescent device according to claim 10, wherein the phosphorescent organic electroluminescent device further comprises:
an electron injecting layer sandwiched between the second electrode layer and the organic electroluminescent layer,
an electron transporting layer sandwiched between the electron injecting layer and the organic electroluminescent layer,
a hole injecting layer sandwiched between the first electrode layer and the organic electroluminescent layer,
a hole transporting layer sandwiched between the hole injecting layer and the organic electroluminescent layer, and
an exciton blocking layer sandwiched between the hole transporting layer and the organic electroluminescent layer.

12. The phosphorescent organic electroluminescent device according to claim 10, wherein the electron injecting layer has a thickness of 0.5 to 1.5 nm, the electron transporting layer has a thickness of 30 to 50 nm, the hole injecting layer has a thickness of 5 to 15 nm, the hole transporting layer has a thickness of 60 to 80 nm, and the exciton blocking layer has a thickness of 2 to 10 nm.
